(19) United States
(12) Patent Application Publication
(10) Pub. No.: US 2025/0084364 A1
(43) Pub. Date: Mar. 13, 2025

(54) PREPARATION METHOD FOR YEAST EXTRACT

(71) Applicants: Guangzhou Aoungo Bio-Tech. Co., Ltd., Foshan (CN); HONNETE & RIGHT BIO-TECH LTD., SURREY (CA)

(72) Inventors: Zhimin WANG, Foshan (CN); Jian PING, Foshan (CN)

(21) Appl. No.: 18/754,093

(22) Filed: Jun. 25, 2024

(30) Foreign Application Priority Data
Sep. 12, 2023 (CN) 202311173517.6

(51) Int. Cl. C12N 1/06; A23L 2/40; A23L 31/15; C12N 1/18

(52) U.S. Cl. CPC C12N 1/063; A23L 2/40; A23L 31/15; C12N 1/185; C12Y 304/22002

(57) ABSTRACT

The present application provides a preparation method for a yeast extract, a corresponding product thereof and an application thereof in soft drinks. The preparation method for a yeast extract of the present application is an autolysis method assisted by adding papain. A chelating agent and an emulsifying agent are added in an autolysis process to adjust sweetness, umami and saltiness of a yeast extract, so as to make the yeast extract more suitable for soft drinks.

### CROSS-REFERENCE TO RELATED APPLICATIONS

This application claims the benefit of Chinese Patent Application No. 202311173517.6, filed on Sep. 12, 2023 in the National Intellectual Property Administration of China, the disclosure of which is incorporated herein by reference in its entirety.

### TECHNICAL FIELD

The present application belongs to the field of beverage and biotechnology, and specifically, the present application provides a preparation method for a yeast extract, a corresponding product thereof and an application thereof in soft drinks.

### BACKGROUND

A yeast extract is a product obtained by using baker's yeast, brewer's yeast, wine yeast, etc. as raw materials (a main strain is Saccharomyces cerevisiae), and degrading proteins and nucleic acids in bacteria by means of autolysis and enzymolysis. The yeast extract has rich nutrients and flavor factors of umami and saltiness. At present, the yeast extract is mainly used for preparing flavorings for freshening (such as chicken, pork and fish flavorings), nutritional supplements and health supplements (using nutrients, glutathione and some antioxidant components thereof), high-quality culture media, etc.

The yeast extract contains a large number of sweet amino acids, such as alanine, proline and threonine, which has a potential to be used as a green and healthy sweetener in the field of beverage. However, as mentioned above, the contents of the flavor factors of umami and saltiness (such as sodium ions, potassium ions, glutamic acid, 5-guanylic acid and 5-inosinic acid) in the yeast extract are high, so that the yeast extract is not suitable for use as a sweetener in soft drinks due to the flavor thereof. At present, a small number of applications thereof in the field of beverage is basically in healthy drinks with a special taste.

How to prepare a yeast extract flavoring agent suitable for soft drinks on the premise of maintaining a basic preparation process and raw materials of the yeast extract is of great significance for expanding the application field thereof and preparing nutritious and healthy soft drink products.

### SUMMARY

Based on the existing method for preparing a yeast extract by autolysis, the applicant has attempted to add a chelating agent and an emulsifying agent to adjust the autolysis system, so as to obtain a yeast extract with strong sweetness and inhibited umami and saltiness, which is more suitable for beverages, especially soft drinks.

On one hand, the present application provides a preparation method for a yeast extract, comprising a step of yeast autolysis, wherein papain is added in the step of autolysis.

Further, the method uses waste brewer's yeast or Saccharomyces cerevisiae powder as a raw material.

Further, the step of autolysis is that: adding water to a yeast paste precipitate or Saccharomyces cerevisiae powder obtained from waste brewer's yeast after washing, debitterization and centrifugation to prepare a yeast suspension of 8%-12% w/v; adding sodium chloride of 1%-3% w/v and papain of 200-500 U/mL, adjusting the pH to 6.0-8.0, and waiting for autolysis at 45° C.-50° C. for 12-36 hours.

Further, the step of autolysis is that: adding water to a yeast paste precipitate or Saccharomyces cerevisiae powder obtained from waste brewer's yeast after washing, debitterization and centrifugation to prepare a yeast suspension of 8%-12% w/v; adding sodium chloride of 2% w/v, papain of 400 U/mL, a chelating agent of 0.1%-0.5% w/v and an emulsifying agent of 0.3%-0.5% w/v, adjusting the pH to 6.5, and waiting for autolysis at 45° C. for 24 hours.

Further, the chelating agent is anhydrous zinc sulfate of 0.3% w/v.

Further, the emulsifying agent is LACTEM of 0.5% w/v.

Further, the method comprises the steps of enzyme killing, ultrafiltration, vacuum concentration and spray drying.

On another hand, the present application provides a yeast extract prepared by the preparation method mentioned above.

On still another hand, the present application provides an application of the yeast extract or the preparation method mentioned above in preparing soft drinks.

Further, the content of the yeast extract in the soft drinks is 0.01%-2% w/v.

The soft drinks mentioned in the present application are those with an alcohol content below a certain threshold, for example 0.5%, i.e., non-alcoholic beverages in the usual sense. Alcohol content standards for soft drinks are related to regulatory standards or different industry practices, and the soft drinks are covered within the protection scope of the present application.

The “yeast extract” and the English abbreviation “YE” in the present application may be used interchangeably to indicate the same meaning.

Sodium acetate and zinc sulfate in the present application are calculated in anhydrous form in experiments, and can be calculated in hydrate form according to corresponding molecular weights in practical application.

### DETAILED DESCRIPTION

### Main Raw Materials and Method

### Yeast:

In order to verify and demonstrate universality of the method of the present application, the applicant has used three yeast raw materials: fresh waste brewer's yeast 1 (distillery A), fresh waste brewer's yeast 2 (distillery B) and Saccharomyces cerevisiae (Angel Yeast).

### Enzyme Preparation:

Papain: sunson.

### Metallic Element Analysis:

An AA320N Plus atomic absorption spectrophotometer (INESA) is used to detect the contents of potassium, sodium, zinc, etc. basically according to the method in GB5009 (by taking an average of three parts).

### Amino Acid Analysis:

An LA8080 amino acid analyzer (Hitachi) is used to detect the content of free amino acids basically according to the method in GB5009 (by taking an average of three parts). Umami amino acids: glutamic acid and aspartic acid; Sweet amino acids: alanine, proline and threonine; Bitter amino acids: phenylalanine, valine, histidine, lysine, leucine, isoleucine, methionine and arginine.

### Flavor Nucleotide Analysis:

Colorimetric analysis is carried out basically according to the method in SB/T 10371-2003 (by taking an average of three parts).

### Other Raw Materials:

Sodium acetate, zinc sulfate, disodium EDTA, phospholipid, LACTEM and lactitol are in domestic food grade raw material.

### Sensory Evaluation Criteria:

A sensory evaluation team is composed of 20 employees from the enterprise of the applicant, including production technicians and non-production administrative/sales personnel fifty-fifty. The evaluation criteria are shown in Table 1 below:

TABLE 1
Evaluation score chart

| Indexes | Description | Scores |
|---|---|---|
| Taste | Acceptant sweet and sour tastes | 7-10 |
| | Good sweet and sour tastes with | 4-6 |
| | other flavor interference | |
| | Mixed tastes giving a bad feeling | 1-3 |
| Fragrance | Built-in fragrance of raw | 7-10 |
| | materials, which is pleasant | |
| | Slight yeasty | 4-6 |
| | Obvious yeasty, masking the | 1-3 |
| | taste of raw materials | |
| Overall | Satisfied | 7-10 |
| preference | Generally satisfied but subject | 4-6 |
| degree | to further adjustment | |
| | Unsatisfied | 1-3 |

### Embodiment 1 Basic Preparation Method for Yeast Extract

After preliminary experiments, a basic preparation method for a yeast extract is determined as follows:

For waste brewer's yeast: adding water, stirring well, sieving and centrifuging; repeating for 3-4 times to obtain a yeast paste without obvious foam or wine taste; adding sodium bicarbonate weighing for 0.5% of the yeast paste, mixing well, debitterizing for 2 hours, centrifuging and removing supernatant; adding water to the yeast paste precipitate obtained by centrifugation to prepare a yeast suspension of 12% w/v; adding sodium chloride of 2% w/v, papain of 400 U/mL and other optional components, adjusting the pH to 6.5, and waiting for autolysis at 45° C. for 24 hours; performing enzyme killing and centrifugation to obtain a yeast extract solution; and performing ultrafiltration, vacuum concentration and spray drying to obtain a powdery yeast extract.

For Saccharomyces cerevisiae powder: adding water to the Saccharomyces cerevisiae powder to prepare a yeast suspension of 8% w/v; adding sodium chloride of 2% w/v, papain of 400 U/mL and other optional components, adjusting the pH to 7.0, and waiting for autolysis at 45° C. for 24 hours; performing enzyme killing and centrifugation to obtain a yeast extract solution; and performing ultrafiltration, vacuum concentration and spray drying to obtain a powdery yeast extract.

Basic sensory assessments show that the yeast extracts prepared from the three raw materials have the basic flavor and morphology of a yeast extract, and the basic indexes are shown in Table 2 below:

TABLE 2
Basic indexes of yeast extract

| | Waste brewer's | Waste brewer's | Saccharomyces |
|---|---|---|---|
| Raw materials | yeast 1 | yeast 2 | cerevisiae |
| Sodium (mg/g) | 5.62 ± 0.05 | 4.98 ± 0.01 | 5.94 ± 0.01 |
| Potassium (mg/g) | 4.23 ± 0.06 | 4.55 ± 0.09 | 5.13 ± 0.02 |
| Zinc (mg/g) | 7.57 ± 0.02 | 9.24 ± 0.07 | 7.72 ± 0.07 |
| Sweet amino | 11.1 ± 0.2 | 10.7 ± 0.2 | 14.5 ± 0.3 |
| acids (mg/g) | | | |
| Bitter amino | 19.7 ± 0.1 | 22.4 ± 0.2 | 20.7 ± 0.4 |
| acids (mg/g) | | | |
| Umami amino | 1.4 ± 0.1 | 1.8 ± 0.1 | 2.4 ± 0.2 |
| acids (mg/g) | | | |
| Flavor | 2.53 ± 0.09 | 2.84 ± 0.12 | 3.41 ± 0.14 |
| nucleotides | | | |
| (mg/g) | | | |

### Embodiment 2 Addition of Chelating Agent

The applicant has attempted to add a chelating agent into the autolysis system to adjust the presence of salty cations in the product (It is generally believed that saltiness is produced by certain cations on ion channels in cell membranes of gustatory cells. Only part of the results with relatively obvious effects are shown, and dosage screening experiments are not shown).

TABLE 3
Effects after addition of chelating agent

| | Waste brewer's | Waste brewer's | Waste brewer's |
|---|---|---|---|
| | yeast 1 | yeast 1 | yeast 1 |
| | (Anhydrous | (Anhydrous | (Disodium |
| | sodium acetate | zinc sulfate | EDTA |
| Preprocessing | of 0.2% w/v | of 0.3% w/v | of 0.2% w/v |
| methods | suspension) | suspension) | suspension) |
| Sodium (mg/g) | 6.21 ± 0.03 | 3.75 ± 0.07 | 5.92 ± 0.06 |
| Potassium (mg/g) | 3.23 ± 0.04 | 2.72 ± 0.06 | 2.95 ± 0.12 |
| Zinc (mg/g) | 6.97 ± 0.11 | 9.67 ± 0.03 | 7.87 ± 0.08 |
| Sweet amino | 10.9 ± 0.4 | 12.1 ± 0.1 | 11.5 ± 0.1 |
| acids (mg/g) | | | |
| Bitter amino | 20.6 ± 0.3 | 19.4 ± 0.2 | 21.7 ± 0.5 |
| acids (mg/g) | | | |
| Umami amino | 1.3 ± 0.2 | 1.4 ± 0.1 | 1.4 ± 0.1 |
| acids (mg/g) | | | |
| Flavor | 2.66 ± 0.12 | 2.74 ± 0.05 | 2.47 ± 0.11 |
| nucleotides | | | |
| (mg/g) | | | |

The results show that although sodium acetate and disodium EDTA can reduce the content of potassium ions, the content of sodium ions is significantly increased due to the introduction of the sodium ions by sodium acetate and disodium EDTA. In contrast, zinc sulfate can significantly reduce the contents of sodium and potassium ions, while increasing the content of zinc ions which are beneficial for taste bud function.

Similar effects are also achieved using waste brewers yeast 2 and Saccharomyces cerevisiae as raw materials, with content of sodium ions reduced by about 34% and 29%, content of potassium ions reduced by about 35% and 36%, content of zinc ion increased by about 31% and 28%, and contents of other components changed insignificantly.

### Embodiment 3 Addition of Emulsifying Agent

In order to further adjust the contents of amino acids and flavor nucleotides, the applicant has attempted to further add an emulsifying agent into the autolysis system (Only part of the results with relatively obvious effects are shown, and type and dosage screening experiments are not shown).

TABLE 4
Effects after addition of emulsifying agent

| | Waste brewer's | Waste brewer's | Waste brewer's |
|---|---|---|---|
| | yeast 1 | yeast 1 | yeast 1 |
| | (Anhydrous | (Anhydrous | (Anhydrous |
| | zinc sulfate | zinc sulfate | zinc sulfate |
| | of 0.3% w/v | of 0.3% w/v | of 0.3% w/v |
| | suspension + | suspension + | suspension + |
| | phospholipid | LACTEM | lactitol |
| Preprocessing | of 0.5% w/v | of 0.5% w/v | of 0.3% w/v |
| methods | suspension) | suspension) | suspension) |
| Sodium (mg/g) | 3.66 ± 0.03 | 3.79 ± 0.06 | 3.85 ± 0.02 |
| Potassium (mg/g) | 2.89 ± 0.07 | 2.66 ± 0.12 | 2.52 ± 0.03 |
| Zinc (mg/g) | 9.72 ± 0.02 | 9.92 ± 0.18 | 10.05 ± 0.09 |
| Sweet amino | 11.7 ± 0.6 | 15.2 ± 0.1 | 10.4 ± 0.3 |
| acids (mg/g) | | | |
| Bitter amino | 21.4 ± 0.4 | 16.7 ± 0.3 | 19.8 ± 0.3 |
| acids (mg/g) | | | |
| Umami amino | 1.4 ± 0.2 | 1.1 ± 0.1 | 1.3 ± 0.1 |
| acids (mg/g) | | | |
| Flavor | 2.54 ± 0.09 | 2.52 ± 0.06 | 2.48 ± 0.11 |
| nucleotides | | | |
| (mg/g) | | | |

The results show that the addition of the emulsifying agent has an obvious influence on the autolysis process, for example, the contents of flavor nucleotides are generally reduced and the contents of amino acids are changed; among which, the adjustment effect of LACTEM is relatively ideal—the contents of umami and bitter amino acids are slightly reduced, while the content of sweet amino acids is increased (Similar trends are also achieved using waste brewers yeast 2 and Saccharomyces cerevisiae as the raw materials, and the content of sweet amino acids is even increased by about 40%).

### Embodiment 4 Evaluation Results of Simulated Beverages

Simple simulated beverages are prepared by using the yeast extract of 0.1% w/v and acesulfame of 0.05% w/v, in combination with apple juice of 30% v/v or an elderberry extract of 3% w/v (without adding complex auxiliary components to fully characterize the influence of the yeast extract), and are evaluated according to the method mentioned above. The scores are shown in the table below:

TABLE 5
Evaluation results

| | Apple juice | Elderberry |
|---|---|---|
| Raw Materials and | beverage | beverage |
| autolysis processing | (total score) | (total score) |
| Yeast extract directly prepared | 340 | 385 |
| from waste brewer's yeast 1 | | |
| Waste brewer's yeast 1 + | 420 | 477 |
| anhydrous zinc sulfate of | | |
| 0.3% w/v suspension | | |
| Waste brewer's yeast 1 + | 485 | 510 |
| anhydrous zinc sulfate of | | |
| 0.3% w/v suspension + | | |
| LACTEM of 0.5% w/v | | |
| suspension | | |
| Yeast extract directly prepared | 309 | 339 |
| from Saccharomyces cerevisiae | | |
| Saccharomyces cerevisiae + | 433 | 447 |
| anhydrous zinc sulfate of 0.3% | | |
| w/v suspension + LACTEM | | |
| of 0.5% w/v suspension | | |

The results show that adding the chelating agent and/or emulsifying agent during the autolysis process can improve the flavoring effect of the yeast extract in soft drinks; on the whole, the yeast extract is more suitable for refreshing drinks such as peppermint drinks, and the yeast extract prepared from waste brewer's yeast is better.

1. A preparation method for a yeast extract, wherein the method comprises a step of yeast autolysis, wherein papain is added in the step of autolysis, wherein the step of yeast autolysis comprises:
adding water to a yeast paste precipitate or Saccharomyces cerevisiae powder obtained from waste brewer's yeast after washing;
preparing a yeast suspension of 8%-12% w/v by debitterization and centrifugation; and
adding sodium chloride of 2% w/v, papain of 400 U/mL, anhydrous zinc sulfate of 5% w/v and LACTEM of 0.5% w/v, adjusting the pH to 6.5, and autolyzing at 45° C. for 24 hours.

2. (canceled)

3. (canceled)

4. (canceled)

5. (canceled)

6. (canceled)

7. The method according to claim 1, wherein the method further comprises the steps of enzyme killing, ultrafiltration, vacuum concentration and spray drying.

8. (canceled)

9. The method according to claim 1, wherein the method is used in preparing soft drinks.

10. The method according to claim 9, wherein the content of the yeast extract in the soft drinks is 0.01%-2% w/v.

11. The method according to claim 7, wherein the method is used in preparing soft drinks.

12. The method according to claim 11, wherein the content of the yeast extract in the soft drinks is 0.01%-2% w/v.
